(19) United States
(12) Patent Application Publication
(10) Pub. No.: US 2006/0142632 A1
(43) Pub. Date: Jun. 29, 2006

(54) Systems and methods for removing plaque from a blood vessel

(76) Inventor: Attila Meretei, Fremont, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/025,236

(22) Filed: Dec. 29, 2004

(51) Int. Cl. A61N 2/00

(52) U.S. Cl. 600/12

(57) ABSTRACT

Systems and methods for removing accumulated plaque in a blood vessel of a patient. Ferrofluids are introduced locally via a catheter, a micro-catheter, or intravenously to the bloodstream of a patient. The ferrofluids are magnetically manipulated or moved throughout the blood vessels of the patient by an external magnetic field generator until the intended accumulated plaque is broken up and removed. The external magnetic field generator, which can be stationary or portable, creates a vortex, high velocity jets or other motion within the ferrofluids, by moving or rotating at least one magnet provided within the magnetic field generator. The vortex, high velocity jets or other motion of the ferrofluids, are used to break-up and remove the accumulated plaque from the blood vessel and to polish the interior surface of the blood vessel after the plaque has been removed therefrom. Drugs or abrasive particles, or both, may be incorporated with the ferrofluids and delivered to the bloodstream to help break-up and remove the accumulated plaque as well. Upon removal of the plaque and polishing of the blood vessel, magnetic components of the ferrofluids may remain in the patient or may be recaptured and magnetically removed from the bloodstream.

### BACKGROUND OF THE INVENTION

1. Field of the Invention

The invention generally relates to systems and methods for removing plaque from a blood vessel. More specifically, the invention relates to systems and methods for delivering and manipulating ferrofluids in the bloodstream of the patient to remove plaque from the blood vessel.

2. Discussion of the Related Art

Plaque formation begins as fatty streaks or deposits on the inner surface of a blood vessel, such as on the inner surface of arterial walls. Over time, the fat deposits accumulate and grow, narrowing the lumen of the blood vessel and hardening the blood vessel wall due to various depositions occurring within the plaque such as lipids, cholesterol, and calcium salts. The narrowing and hardening of the blood vessels can have dramatic effects on blood pressure and blood flow within the blood vessels.

For example, when blood flow is reduced due to the narrowing of a blood vessel, organs may be deprived of oxygen intended to be carried in greater volume through the blood vessels. If the oxygen supply to the heart muscle is reduced sufficiently, a heart attack can occur. If the oxygen supply to the brain is reduced sufficiently, a stroke can occur. Likewise, where hardening of the blood vessel walls occur due to plaque accumulations, the ability of the blood vessel to distend is reduced resulting in an increase of blood pressure in the blood vessels. As blood pressure increases, the heart has to work harder to pump blood, undesirably causing the heart to enlarge.

In the past, surgical procedures have been used to remove accumulations of plaque from a blood vessel. For example, as shown in FIG. 1, the carotid artery 1 supplies blood and oxygen to the brain. As shown in FIGS. 2a-2c, where the carotid artery 1 has had an accumulation of plaque 2, a carotid endarterectomy has often been performed to surgically open a section of the carotid artery 1 and remove the plaque 2 from the carotid artery. The carotid artery is then sutured closed (FIG. 2c) and blood flow is restored through the carotid artery 1. The carotid endarterectomy has been widely used as a way to reduce the risk of stroke by removing the plaque accumulation from the carotid artery. While often effective, the carotid endarterectomy risks causing the very stroke the procedure is intended to avoid if a cerebral embolism, i.e., a blood clot that breaks loose and travels to the brain, were to occur during the procedure.

Of course, accumulations of plaque can occur almost anywhere in the body. Mechanically compressing the plaque using a balloon angioplasty to widen the lumen within the blood vessel has also been used to treat plaque accumulations in blood vessels as well. Likewise, bypass surgery has been used to graft another vessel through which blood flow may occur to avoid the plaque obstructed blood vessel altogether. Neither of these methods remove the plaque accumulations from the blood vessels however.

Alternatively, drugs such as statins are often ingested or otherwise administered to patients to reduce, or minimize the likelihood of, plaque accumulations in blood vessels. Such drugs, however, may deplete patients of other important substances over time. All of the treatment alternatives practiced to date have drawbacks therefore.

In view of the above, a need exists for systems and methods that more easily and safely treat and remove accumulations of plaque in a blood vessel.

### SUMMARY OF THE INVENTION

The systems and methods of the invention introduce ferrofluids into the bloodstream of a patient and magnetically manipulate the ferrofluids in order to break up and remove plaque accumulations therewithin. In some embodiments of the systems and methods of the invention, the ferrofluids are introduced locally to a targeted blood vessel using a catheter or micro-catheter. More specifically, a micro-catheter is used to deliver ferrofluids locally to smaller blood vessels, whereas a catheter is used to deliver ferrofluids locally to larger blood vessels. The ferrofluids are then magnetically manipulated to remove the accumulated plaque. In other embodiments, the ferrofluids are intravenously introduced to the bloodstream of the patient. The intravenously introduced ferrofluids are magnetically moved to the site of the accumulated plaque and then further magnetically manipulated to break-up and remove the accumulated plaque. Upon removal of the accumulated plaque, the ferrofluids may be further magnetically manipulated to polish the interior surface of the blood vessel.

In some embodiments, the ferrofluids incorporate abrasive particles that are manipulated along with the ferrofluids to break-up the accumulated plaque in the bloodstream. In other embodiments, the ferrofluids incorporate anti-plaque drugs that are manipulated along with the ferrofluids to break-up the accumulated plaque in the bloodstream. In still other embodiments, the ferrofluids incorporate a combination of abrasive particles and an anti-plaque drug that are manipulated along with the ferrofluids to break-up the accumulated plaque in the bloodstream.

In those embodiments where a micro-catheter is used to introduce the ferrofluids into the bloodstream, the micro-catheter may include at least one sensor at a tip of a guidewire, for example. The at least one sensor helps identify conditions pertaining to the accumulated plaque site and the activity of the ferrofluids at that site in particular. To this end, the at least one sensor may identify conditions such as pressure, temperature, flow, shaft deflection or the like to indicate how the ferrofluids are acting at the accumulated plaque site within the blood vessel. Based on the data sensed by the at least one sensor, the magnetic field, or the position of the patient relative to the magnetic field, may be altered in order to more appropriately manipulate the ferrofluids to break-up and remove the accumulated plaque.

The systems and methods of the invention further provide a magnetic field generator. The magnetic field generator is external of the patient and is used to manipulate or move the ferrofluids within the bloodstream of the patient. The magnetic field generator may induce a vortex, high velocity jets or other motion, in the ferrofluids introduced into the bloodstream of the patient. The magnetically induced vortex, high velocity jets or other motion in the ferrofluids, breaks-up and removes the accumulated plaque in the targeted blood vessel. After the accumulated plaque is removed and the interior surface of the blood vessel polished, the ferrofluids may remain in the patient for eventual consumption by naturally occurring phagocytotic cells, or the ferrofluids may have magnetic components of the ferrofluids magnetically recaptured and removed from the bloodstream using the catheter or micro-catheter, for example.

In some embodiments, the magnetic field generator comprises a tubular member into which the patient is placed. The magnetic field generator in this instance is similar to an MRI or CT scanner system, whereby the patient lies prone on a movable table that is transportable into and out of the tubular member. The tubular member according to this embodiment of the systems and methods of the invention further comprises a movable collar having at least one magnet circumferentially arranged about a portion of the tubular member. The collar having at least one magnet surrounds the body part of the patient having the accumulated plaque. When stationary, the collar having at least one magnet provides a magnetic field sufficient to concentrate intravenously delivered ferrofluids at the intended accumulated plaque site. When rotated, or otherwise moved, the collar having at least one magnet provides a magnetic field sufficient to induce the vortex, high velocity jets or other motion of the ferrofluids at the accumulated plaque site that is used to break-up and remove the accumulated plaque. Of course, independent magnets could instead be used to concentrate the intravenously delivered fluids at the intended accumulated plaque site.

In still other embodiments, the magnetic field generator is a more portable system transportable in emergency vehicles, for example. The portable magnetic field generator comprises a smaller scale tubular member into which the body part having the accumulated plaque is placed. The portable tubular member also comprises a movable collar having at least one magnet that surrounds the accumulated plaque site of the patient when the body part is placed within the portable tubular member. As in the larger scale tubular member, the stationary collar having at least one magnet provides a sufficient magnetic field to concentrate the intravenously delivered ferrofluids at the intended accumulated plaque site, whereas rotation of the collar having the at least one magnet provides a sufficient magnetic field to induce the vortex, high velocity jets or other motion of the ferrofluids that breaks-up and removes the accumulated plaque similar to as described above. The portable tubular member is ideally sufficiently lightweight that it can be managed by a single emergency or other medical professional and placed around the intended body part with minimal movement of the patient.

The systems and methods of the invention thus provide a low profile delivery system for delivering ferrofluids to the bloodstream, whereby the ferrofluids are easily manipulated within a vessel. Rigid mechanical components are not required to be introduced to the bloodstream or to penetrate through the accumulated plaque in a blood vessel. Unintended damage to blood vessels or other organs is minimized as a result.

The systems and methods of the invention simplify the treatment and removal of accumulated plaque from a blood vessel and can require less training for a medical professional administering the ferrofluids to a patient. Where the ferrofluids are introduced intravenously to the bloodstream of a patient, emergency medical personnel or front-line hospitals, rather than specialized stroke and neuro-vascular oriented medical centers, may more easily administer the ferrofluids to a patient. The ready access of such emergency and front-line hospitals equipped with the systems and methods of the invention can minimize the detrimental impact an accumulated plaque can have on a patient. Thus, the systems and methods of the invention provide a safer, simpler, and easier manner of treating and removing accumulated plaque within a blood vessel of a patient.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates generally a carotid artery with an accumulation of plaque.

FIGS. 2a-2c illustrate various stages of a carotid endarterectomy practiced in the art.

FIG. 3 illustrates a blood vessel with an accumulation of plaque therein.

FIG. 4 illustrates a carotid artery with an accumulation of plaque therein.

FIG. 5 illustrates lower limb blood vessels in which accumulation of plaque may occur.

FIGS. 6a-6c illustrate various ferrofluid nanoparticles according to the invention.

FIG. 7 illustrates a micro-catheter introducing ferrofluids locally to a blood vessel in a patient according to the invention.

FIG. 8 illustrates a magnetic field generator according to one aspect of the invention.

FIG. 9 illustrates a patient subjected to the magnetic field generator of FIG. 8 according to the invention

FIGS. 10a-10c illustrate various stages of the vortex created by the ferrofluids breaking-up and removing accumulated plaque according to the invention.

FIG. 11 illustrates a portable magnetic field generator according to another aspect of the invention.

FIG. 12 illustrates a patient subject to the portable magnetic field generator of FIG. 11 according to the invention.

FIG. 13 illustrates a micro-catheter guide wire tip having sensors incorporated therewith according to another aspect of the invention.

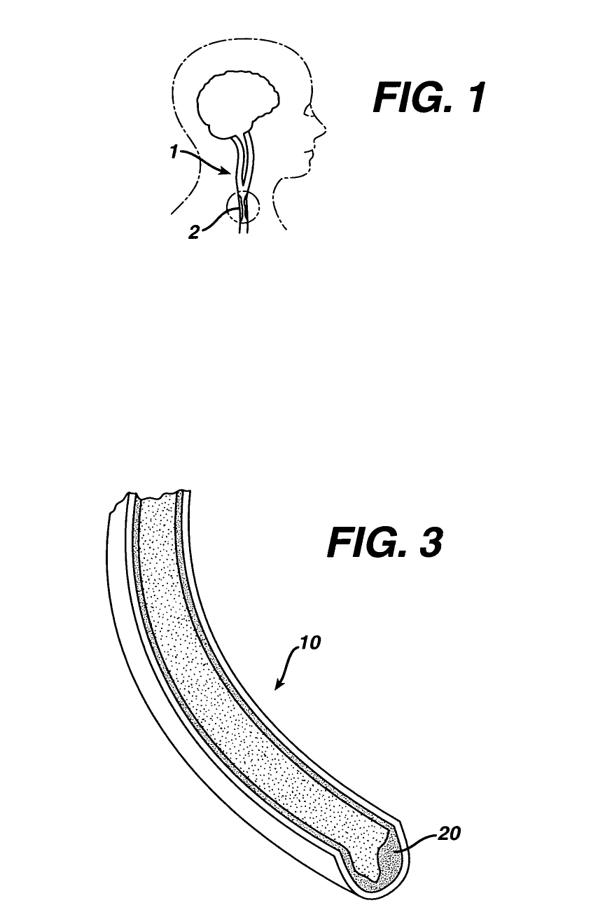

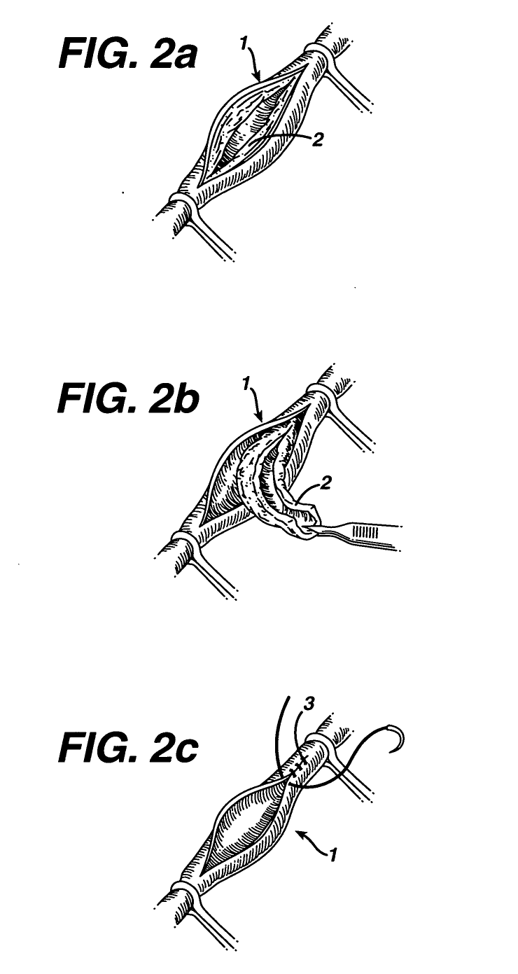

### DETAILED DESCRIPTION OF THE INVENTION

FIG. 3 illustrates an exemplary blood vessel 10 of the vasculature of a human anatomy. The blood vessel 10 is shown as having an accumulation of plaque 20 extending along an interior surface of the blood vessel. The accumulated plaque 20 narrows the blood-flow passageway through the blood vessel 10 increasing the risk of hypoxia, stroke or other undesirable maladies and conditions in a patient. The accumulation of plaque 20 may occur anywhere in the vasculature of the human anatomy, but is often formed in the arterial blood vessels of the neck, heart and the lower limbs of a patient.

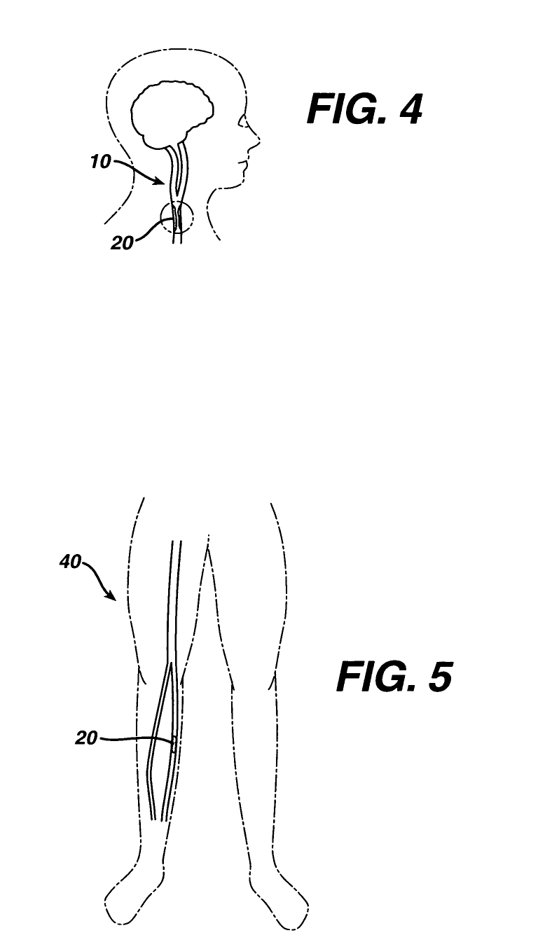

FIG. 4 illustrates accumulations of plaque 20 located in the carotid artery 10, for example. FIG. 5 illustrates accumulations of plaque 20 located in blood vessels of the leg 40, for example. The systems and methods for treating accumulations of plaque according to the invention may be applied to treat accumulated plaque located anywhere in the vasculature of the human anatomy. The artisan should readily appreciate therefore that the accumulations of plaque shown and described herein with respect to either of the carotid artery and blood vessels of the lower limbs are for illustrative purposes only. Similar systems and methods may be used to treat accumulations of plaque at other locations in the vasculature of a patient as well using the systems and methods of the invention.

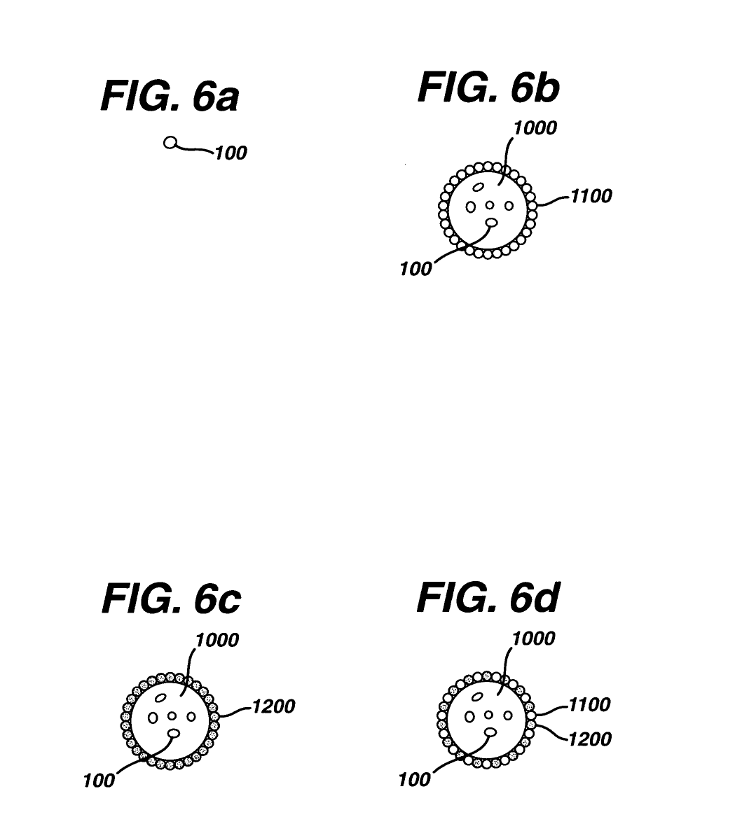

FIGS. 6a-6c illustrate various configurations of a ferrofluid according to the invention. The ferrofluids may be colloids with magnetic particles of nanometer or micron size dispersed in a carrier fluid. The carrier fluid may be water, water-alcohol mixtures, or a variety of hydrocarbons, such as paraffin oil or synthetic esters, for example. The magnetic particles may be comprised of materials such as magnetite or cobalt ferrite, for example. The artisan should appreciate that other materials having similar properties as known in the art are also readily usable as the carrier fluids or magnetic particles, as appropriate, with the systems and methods of the invention.

FIG. 6a more specifically illustrates a single magnetic particle 100 according to the invention. A plurality of magnetic particles 100 in colloid solution within a carrier fluid comprises the ferrofluids that are delivered to the bloodstream of a patient to break-up and remove accumulated plaque according to the invention. Each single magnetic particle 100 is preferably 5-500 nanometers in size and is comprised of bio-compatible material of sufficient radiopacity as to be visible using known fluoroscopy techniques, for example. However, non-radiopaque magnetic particles may also be used if the ferrofluid in the vasculature is visualized by means other than fluoroscopy. Of course, the artisan will appreciate that the ferrofluids may also comprise a combination of different magnetic particles 100 dispersed within the colloid.

FIG. 6b illustrates a ferrofluid droplet 1000 having abrasive particles 1100 added thereto. The abrasive particles 1100 are thus added to the ferrofluid colloid. The abrasive particles 1100 will be displaced to a surface of the ferrofluid droplet 1000 (as shown in FIG. 6b) once the ferrofluid droplet 1000 is exposed to a sufficient magnetic field. Other abrasive particles may also be expelled to the surface of the ferrofluid droplets because of surface characteristics. Such surface characteristics may include hydrophobic functional groups that would expel the abrasive particle to the surface of a ferrofluid droplet having a hydrophilic carrier fluid. Alternately, abrasive particles having hydrophilic functional groups on their surface would be expelled to the surface of a ferrofluid having a hydrophobic carrier fluid. The abrasive particles may be comprised of a bio-compatible abrasive material, such as beads made of glass, polymers, magnetic or non-magnetic metals, or any other bio-compatible abrasive material known in the art that is able to mix with the ferrofluid droplet 1000.

FIG. 6c illustrates a ferrofluid droplet 1000 having drug carrier particles 1200 dispersed therein and being impregnated with an anti-plaque drug, some of which may be expelled to the surface of the ferrofluid droplet 1000. The anti-plaque drug may comprise a plaque reducing or plaque formation inhibiting drug as known or later developed in the art. The anti-plaque drug may be bound to the surface of the magnetic particles 100 dispersed within the ferrofluids. Alternatively, the anti-plaque drug may be bonded to the abrasive particles 1100, may be carried in pores or internal cavities of the magnetic particles 100, or may be added to the colloid of ferrofluids via carrier particles 1200 (FIG. 6d) designed to carry the anti-plaque drug within the ferrofluids.

FIG. 6d illustrates a ferrofluid droplet 1000 having carrier particles 1200 that carry an anti-plaque drug. As shown in FIG. 6d, the ferrofluid droplet 1000 also contains abrasive particles 1100, which have been displaced to the surface of the ferrofluid droplet 1000 after exposure to a magnetic field according to the systems and methods of the invention.

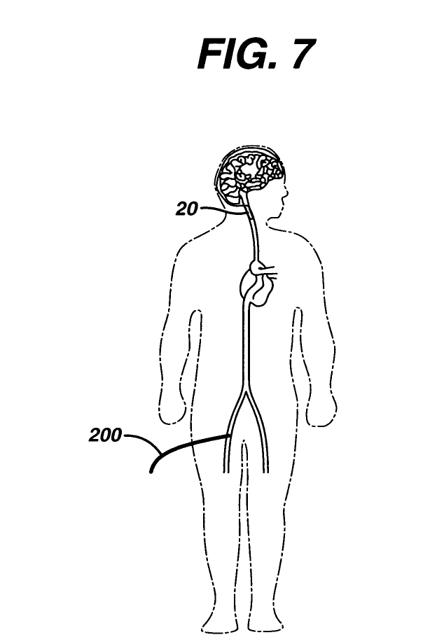

In practice, ferrofluids are delivered to the site of the accumulated plaque in conventional manner using a catheter, a micro-catheter or intravenously. As shown in FIG. 7, wherein a micro-catheter 200 is used to deliver ferrofluids to the carotid artery, for example, the ferrofluids are generally delivered directly to the site of the accumulated plaque 20. On the other hand, when intravenous delivery of the ferrofluids is used (not shown), the ferrofluids are delivered systemically to the bloodstream of the patient and then subsequently magnetically manipulated to the site of the accumulated plaque according to the invention.

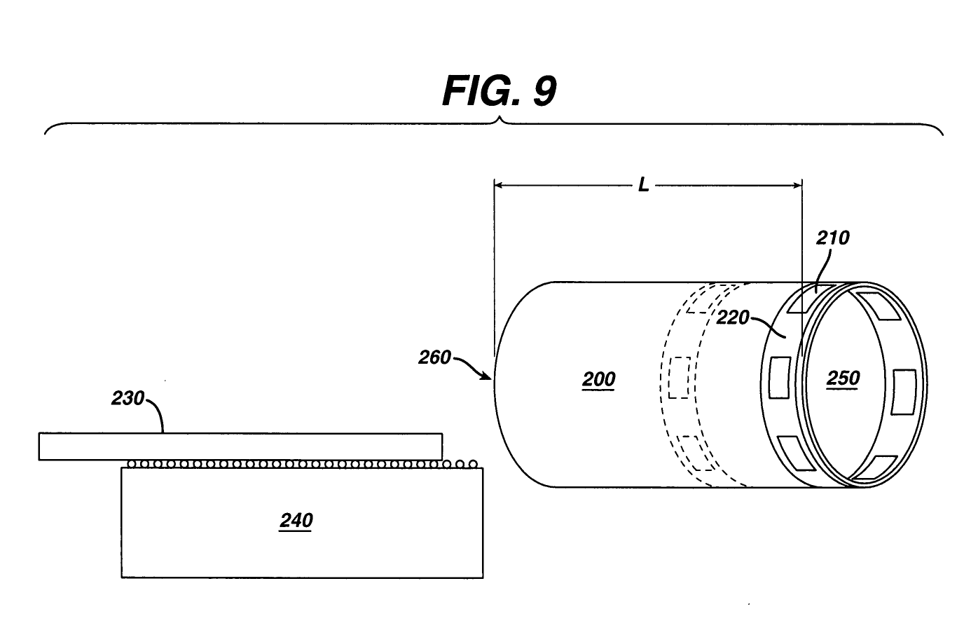

Once the ferrofluids are located at the site of the accumulated plaque, whether by direct catheter or micro-catheter delivery or by indirect intravenous delivery with subsequent magnetic manipulation, the ferrofluids are then subjected to a magnetic field generated by an external magnetic field generator, as will be discussed in more detail below with respect to FIGS. 9-12. The external magnetic field generator externally encircles a body part of the patient whereat the accumulated plaque is located. By moving or rotating the magnetic field, the ferrofluids within the blood vessel of the encircled body part are also rotated or moved.

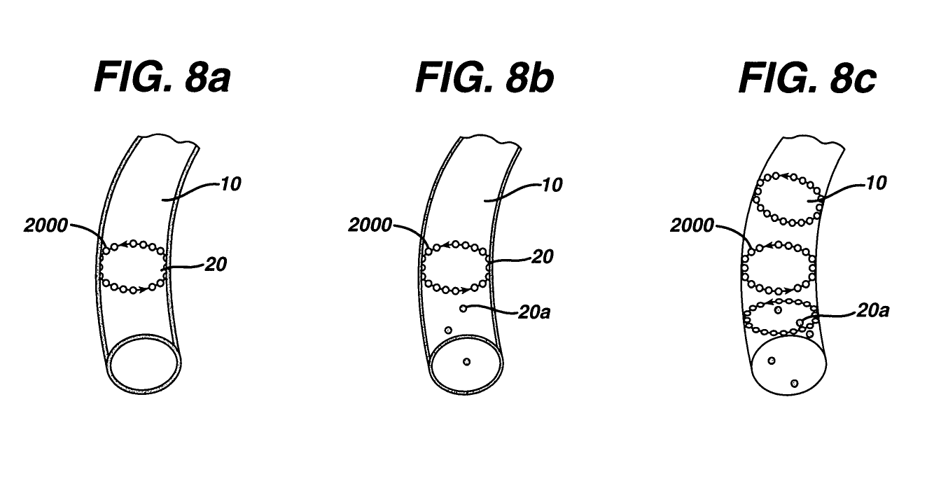

FIGS. 8a-8c illustrate various stages of rotating the ferrofluids at the site of the accumulated plaque 20 within a blood vessel 10. For example, FIG. 8a illustrates the ferrofluids being rotated in the blood vessel 10. By rotating the external magnetic field sufficiently the ferrofluids are induced to form a vortex 2000 within the blood vessel 10. Ideally, as shown in FIGS. 8a-8c, the vortexed ferrofluids 2000 rotate along the inner surface of the blood vessel 10 to break-up and remove the accumulated plaque 20 from the blood vessel 10. The ferrofluids may instead be induced by the magnetic field to produce high velocity jets or other motion in the ferrofluids to break-up and remove the accumulated plaque, although the inducement and use of the vortex 2000 is described in greater detail herein.

In FIG. 8a, for example, the vortex 2000 of ferrofluids has just begun and the accumulated plaque 20 is largely intact along the interior surface of the blood vessel 10. In FIG. 8b, the vortex of ferrofluids 100 has taken place for awhile and has broken-up or otherwise detached portions of the accumulated plaque 20 from the blood vessel 10. Remnants 20a of the plaque 20 broken-up by the vortexed rotation of the ferrofluids 100 are evident in FIG. 8b, for example. The swath or width of plaque broken-up by the vortexed ferrofluids is generally that portion of accumulated plaque 20 contacted directly by the ferrofluids 100. The wider the band of ferrofluids, the more accumulated plaque is contacted and removed at one time. The ferrofluids may be magnetically manipulated to contact various portions of the accumulated plaque along the interior surface of the blood vessel 10 until the entire area of accumulated plaque has been contacted and removed by the ferrofluids. Ideally, the remnants 20a that occur as a result of the vortexed ferrofluids action on the accumulated plaque 20 are generally dispersed through the blood vessel 10 without event.

In FIG. 8c the ferrofluids 100 have been manipulated to various locations along the blood vessel and the accumulated plaque 20 has been removed therefrom by the vortexed ferrofluids 10, leaving only the loosened remnants 20a of the accumulated plaque for dispersal through the bloodstream. Although the rotation of the vortex 2000 shown in FIGS. 8a-8c is counter-clockwise, the artisan will appreciate that clockwise, or other, rotational directions are also usable according to the systems and method of the invention.

Where the ferrofluids 1000 used have magnetic particles 100 but are devoid of abrasives 1100 or anti-plaque drugs, the vortexed ferrofluids 2000 may simply detach the accumulated plaque 20 from the blood vessel 10, rather than fully breaking up the accumulated plaque. Thereafter, the detached plaque 20 may be mechanically extracted from the blood vessel using a catheter or micro-catheter in conventional manner. The risks of the additional extraction procedure are self-evident, but such a drug-less procedure minimizes drug-induced side-effects that can occur when anti-plaque drugs are used to treat an accumulation of plaque.

Where the ferrofluids 1000 have magnetic particles 100 and are impregnated with abrasive particles 1100, the abrasive particles 1100 work in combination with the vortex 2000 to break-up and remove the accumulated plaque 20, ideally without need for mechanically extracting any part of the plaque. The absence of the additional extraction procedure minimizes risk of puncture or other damage to the blood vessel in which the accumulated plaque is located. The absence of an anti-plaque drug minimizes or eliminates the risk of side-effects associated with the use of such drugs.

Where the ferrofluid droplets 1000 with magnetic particles 100 are impregnated with an anti-plaque drug via carrier particles 1200, the anti-plaque drug work in combination with the vortex 2000 to break-up and remove the accumulated plaque 20, ideally without need to mechanically extract any portion of the thrombus. Though the risk of side-effects is present due to the use of the anti-plaque drug in this instance, the risks attendant with the additional extraction procedure of a drugless procedure are minimized.

Of course, the artisan will appreciate that where a combination of abrasive particles 1100 and an anti-plaque drug is used with the ferrofluid droplets 1000, the combination works with the vortex 2000 to break-up and remove the accumulated plaque 20, ideally also without the need for mechanical extraction of the plaque. Thus, the use of such a combination would minimize at least the risks associated with the additional extraction procedure.

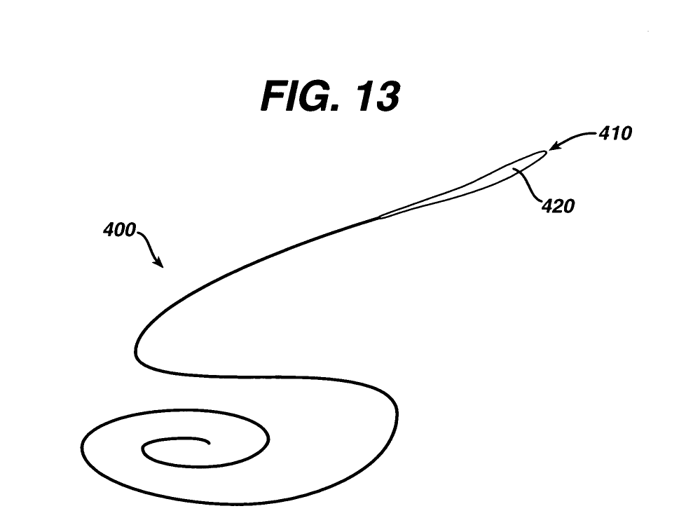

Once the accumulated plaque 20 is removed, the components of the ferrofluids that are magnetic, i.e., the magnetic particles 100, the abrasive particles 1100 if made of magnetic materials, and any carrier particles 1200 if made of magnetic materials, may either remain in the patient's bloodstream, or may be magnetically recaptured and removed from the body using a catheter or micro-catheter by directing the magnetic components out of the bloodstream through the catheter or micro-catheter, as the case may be. The catheter or micro-catheter in this instance could include a guide-wire having a magnetic tip (FIG. 13), for example,.that would guide the magnetic components of the ferrofluids through and out of the blood vessel 10, via the catheter or micro-catheter, as the guide-wire is extracted from the blood vessel in conventional manner.

Referring to FIG. 9, a magnetic field generator system is shown. The magnetic field generator system comprises a tubular member 200, at least one magnet 210, a collar 220, a movable table 230 and a base 240. The at least one magnet 210 is placed in the collar 220 positioned along a circumference of the tubular member 200. The collar 220 is movable or rotatable. Of course, the artisan should readily appreciate that the at least one magnet 210, shown as a plurality of magnets 210 in FIG. 9, could as well be comprised of a single magnetic band within the collar 220. Moreover, where provided, the plurality of magnets may be arranged on or within the collar 220 in a variety of configurations.

As shown in FIG. 9, the collar 220 is positioned near a first end 250 of the tubular member. The collar 220 is translatable along the length L of the tubular member 200 such that the collar 220 can be positioned anywhere along the length L of the tubular member 200 between the first end 250 and a second end 260. For example, the collar 220 is shown in dashed lines about midway between the first end 250 and the second end 260 in FIG. 9 as well. Regardless of the translational position of the collar 220 along the tubular member 200, the collar 220 is movable or rotatable about the circumference of the tubular member. In this manner, the magnetic field generated by the at least one magnet 210 in the collar 220 is applied to the patient received within the tubular member 200. Furthermore, the collar 220 may be tilted, allowing the collar to rotate about any axis within three-dimensional space.

Referring still to FIG. 9, the movable table 230 is movably mounted to a base 240. The table 230, for example, may move into and out of the tubular member 200 in order to position a patient in the tubular member 200 for appropriate orientation relative to the at least one magnet 210 and collar 220.

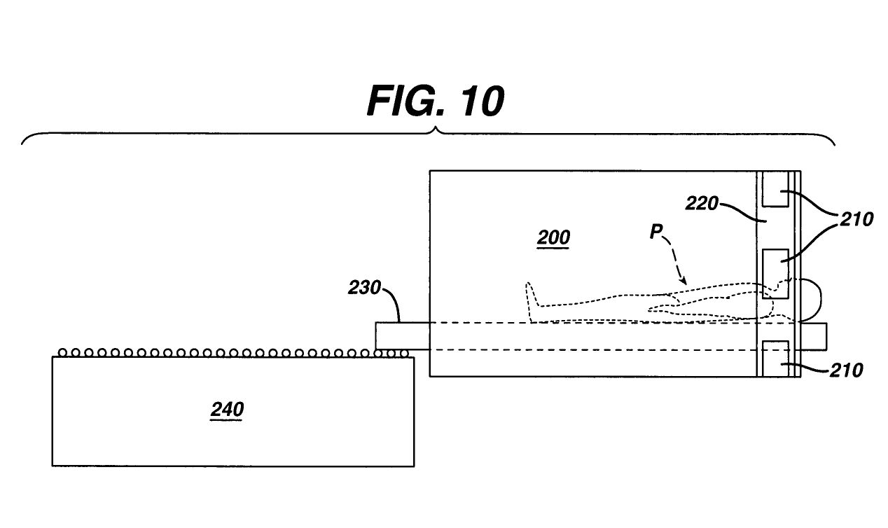

FIG. 10 illustrates more specifically a patient P positioned on the table 230 within the tubular member 200. The neck of the patient P, for example, is oriented generally in line with the at least one magnet 210 in the collar 220. Such an orientation of the patient's neck in this manner is contemplated when an accumulation of plaque in a carotid artery of a patient is sought to be treated using the systems and methods of the invention. In practice, the collar 220 is moved or rotated to generate a magnetic field about the neck of the patient P. The magnetic field induces the vortex 2000, high velocity jets or other motion, in the ferrofluids previously delivered to the patient. The accumulated plaque is then broken-up and removed by the vortexed ferrofluids as described above according to the type of ferrofluid droplets 1000 used. Rotation or movement of the collar 220 and the at least one magnet 210 is stopped, and the patient is removed from the tubular member after the accumulated plaque has been broken-up and removed.

A conventional switch (not shown) may be used with the magnetic field generator in order to move or rotate the collar 220 when desired. Likewise, a conventional switch and moving means, such as a belt drive, rollers, glide systems or combinations thereof, may be used to move the table 230 along the base 240 and into and out of the tubular member 200 when desired. The magnetic field generator is otherwise powered by conventional means.

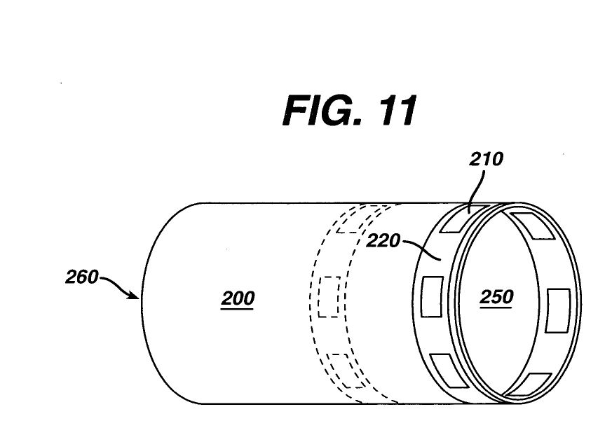

FIG. 11 illustrates a smaller scale portable magnetic field generator system according to the systems and methods of the invention, wherein like reference numbers are used to identify like features. As shown in FIG. 11, the magnetic field generator comprises a tubular member 200, at least one magnet 210, and a collar 220. The tubular member 200 has a first end 250 and a second end 260. The at least one magnet 210 is placed in the collar 220 positioned along a circumference of the tubular member 200. Of course, the artisan should readily appreciate that, as with the magnetic field generator of FIG. 9, the at least one magnet 210, shown as a plurality of magnets 210 in FIG. 11, could as well be comprised of a single magnetic band within the collar 2200. The collar 220 is similarly movable or rotatable as in earlier described embodiments.

The magnetic field generator of FIG. 11 and operation thereof is generally the same as that described with reference to the magnetic field generator of FIG. 9 except that the tubular member 200 of FIG. 11 receives only a body part of a patient rather than the entire patient P (FIG. 10). Thus, the dimensions of the tubular member 200 and associated components of FIG. 11 are proportionately diminished from those of the tubular member 200 and associated components of FIG. 9. The artisan should readily appreciate the dimensional differences, which are omitted herein for brevity.

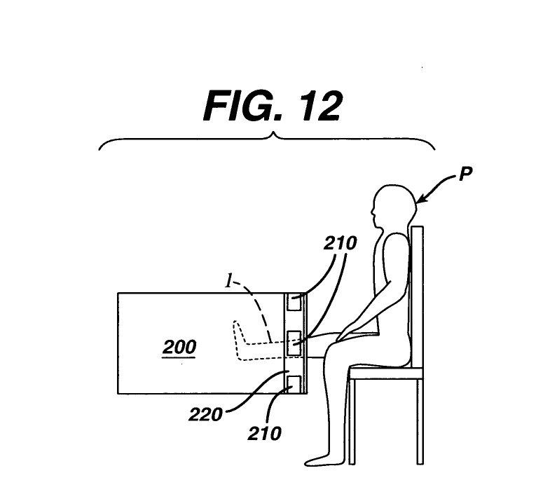

FIG. 12 illustrates a lower limb I of a patient P being received within the tubular member 200 of the magnetic field generator of FIG. 11. Such lower limb I would be received within the tubular member 200 when an accumulation of plaque exists in the lower limb. The collar 220 and the at least one magnet 210 are then rotated to induce the vortex 2000 in the ferrofluids within the blood vessel whereat the accumulated plaque is located. The accumulated plaque is thus broken-up and removed in a manner consistent with the type of ferrofluid droplets 1000 used. The magnetic field is then terminated. After break-up and removal of the accumulated plaque, the ferrofluids either remain in the bloodstream for eventual consumption by naturally occurring macrophages, or the magnetic components of the ferrofluids are removed from the bloodstream using a magnetic tipped guide-wire and micro-catheter in conventional manner as discussed above. Rotation or movement of the collar 220 and the at least one magnet 210 is stopped, and the patient's body part is removed from the tubular member 200 after the plaque has been broken-up and removed.

Ideally, the portable magnetic field generator is transportable using a transport device, such as a conventional dolly-like apparatus, for example, similar to the manner in which oxygen tanks are commonly transported. Preferably, the transport device would include a power supply system to which the magnetic field generator could be connected. Of course, the artisan should readily appreciate that, where provided, the power supply system would provide sufficient power to generate a magnetic field of sufficient gradient to induce the vortex of the ferrofluids used to break-up and remove the accumulated plaque. Alternatively, the portable magnetic field generator could be powered by other conventional non-portable means.

FIG. 13 illustrates a guide-wire 400 for use with a conventional catheter or micro-catheter according to the systems and methods of the invention. The guide-wire 400 comprises a magnetic tip 410 and at least one sensor 420 at its tip, but is otherwise conventional. The at least one sensor 420 may be inserted through the catheter or micro-catheter with the ferrofluids 100 to the site of the accumulated plaque. The at least one sensor 420 may be used to measure pressure, temperature, guide-wire tip deflection, or other conditions occurring at the site of the accumulated plaque in order to indicate how the ferrofluids are performing. For example, where the at least one sensor 420 is a tip deflection sensor, the sensor 420 will detect the amount or angle of deflection of the guide-wire tip 410 within the blood vessel, wherein the bending or deflection of the guide-wire tip complies with the direction of movement or rotation of the magnetic field generated by the magnetic field generator of FIG. 9 or FIG. 11. If the amount or degree of angular deflection of the tip 410 is insufficient, one can presume that the intended vortex 2000 of the ferrofluids is not being created by the generated magnetic field. The strength, geometry, or gradient of the magnetic field can thus be altered, or the position of the patient can be altered to more appropriately align the site of the accumulated plaque with the magnets, and hence the magnetic field, of the magnetic field generator. Where provided, the magnetic tip 410 of the guide-wire 400 is also usable to magnetically attract and remove the magnetic components of the ferrofluids from the bloodstream of the patient after the accumulated plaque has been removed, as discussed earlier. Still further, the at least one sensor may be provided on a tip of the catheter or micro-catheter. Alternatively, the at least one sensor can simply be omitted.

The various exemplary embodiments of the invention as described hereinabove do not limit different embodiments of the present invention. The material described herein is not limited to the materials, designs, or shapes referenced herein for illustrative purposes only, and may comprise various other materials, designs or shapes suitable for the systems and procedures described herein as should be appreciated by one of ordinary skill in the art.

While there has been shown and described what is considered to be preferred embodiments of the invention, it will, of course, be understood that various modifications and changes in form or detail could readily be made without departing from the spirit or scope of the invention. It is therefore intended that the invention be not limited to the exact forms described and illustrated herein, but should be construed to cover all modifications that may fall within the scope of the appended claims.

What is claimed is:

1. A system for removing accumulated plaque in a blood vessel of a patient, the system comprising:
magnetically manipulable ferrofluids disposed within the blood vessel; and
a magnetic field generator that generates a magnetic field to manipulate the ferrofluids within the blood vessel to remove the accumulated plaque.

2. The system of claim 1, wherein the ferrofluids further comprise magnetic particles dispersed in a carrier fluid within the ferrofluids.

3. The system of claim 2, wherein the carrier fluid is one of a water-based, a water-alcohol-based, or a hydrocarbon-based carrier fluid.

4. The system of claim 3, wherein the carrier fluid is hydrophilic.

5. The system of claim 3, wherein the carrier fluid is hydrophobic.

6. The system of claim 3, wherein the ferrofluids further comprise abrasive particles mixed into the ferrofluids, the abrasive particles displaceable to a surface of the ferrofluids when exposed to a magnetic field.

7. The system of claim 4, wherein the ferrofluids further comprise hydrophobic abrasive particles mixed into the ferrofluids, the hydrophobic abrasive particles displaceable to a surface of the ferrofluids when subject to the hydrophilic carrier fluid.

8. The system of claim 5, wherein the ferrofluids further comprise hydrophilic abrasive particles mixed into the ferrofluids, the hydrophilic abrasive particles displaceable to a surface of the ferrofluids when subject to the hydrophobic carrier fluid.

9. The system of claim 3, wherein the ferrofluids further comprise an anti-plaque drug.

10. The system of claim 9, wherein the anti-plaque drug is bonded to one of the magnetic particles or the abrasive particles, is mixed into the carrier fluids, or is added to the ferrofluids by carrier particles to deliver the anti-plaque drug throughout the ferrofluids.

11. The system of claim 2, wherein the ferrofluids further comprise a combination of abrasive particles and an anti-plaque drug carried with the ferrofluids.

12. The system of claim 1, further comprising:
a catheter or a micro-catheter for delivering the ferrofluids directly to a site of the accumulated plaque within the bloodstream.

13. The system of claim 12, further comprising at least one sensor on or near a tip of the catheter or micro-catheter for determining the activity of the ferrofluids at the site of the accumulated plaque.

14. The system of claim 12, further comprising:
a magnetically tipped guide-wire.

15. The system of claim 14, further comprising;
at least one sensor on the tip of one of the catheter, the micro-catheter, or the guide-wire for determining the activity of the ferrofluids at the site of the accumulated plaque.

16. The system of claim 15, wherein one of the at least one sensor is a deflection sensor to determine the amount of deflection of the guide-wire tip as an indication of the activity of the ferrofluids at the site of the occluding thrombus.

17. The system of claim 16, wherein the at least one sensor further comprises a temperature or pressure sensor.

18. The system of claim 17, wherein a strength, geometry or gradient of the magnetic field is determined based on the data sensed from the at least one sensor.

19. The system of claim 1, wherein the magnetic field generator further comprises:
a tubular member having a first end and a second end;
a collar positionable along an exterior circumference of the tubular member between the first end and the second end of the tubular member;
at least one magnet provided with the collar;
a movable table movably mounted to a base, the movable table oriented to hold the patient and movable into and out of the tubular member with the patient aboard said table; and
means for powering the movable table and the collar.

20. The system of claim 1, wherein the magnetic field generator further comprise:
a tubular member having a first end and a second end;
a collar positionable along an exterior circumference of the tubular member between the first end and the second end of the tubular member;
at least one magnet provided with the collar; and
means for powering the collar, wherein only a body part of the patient is received within the tubular member.

21. The system of claim 19, wherein the collar is at least one of rotatable or movable.

22. The system of claim 20, wherein the collar is at least one of rotatable or movable.

23. The system of claim 19, wherein the at least one magnet is a permanent magnet.

24. The system of claim 19, wherein the at least one magnet is an electromagnet.

25. The system of claim 20, wherein the at least one magnet is a permanent magnet.

26. The system of claim 20, wherein the at least one magnet is an electromagnet.

27. The system of claim 1, further comprising an intravenous delivery of the ferrofluids to the bloodstream.

28. The system of claim 27, wherein the ferrofluids are concentrated to a site of the accumulated plaque prior to the removal of the accumulated plaque by the ferrofluids.

29. The system of claim 1, wherein the ferrofluids comprise a vortex, high velocity jets, or other motion for breaking up and removing the accumulated plaque and polishing an interior surface of the blood vessel upon manipulation by the magnetic field.

30. The system of claim 14, wherein the guide-wire with the magnetic tip further comprises a magnetic re-capture device for re-capturing and directing magnetic components within the ferrofluids through the catheter or micro-catheter to remove the magnetic components of the ferrofluids from the blood vessel.

31. A method for removing accumulated plaque in a blood vessel, the method comprising:
delivering ferrofluids having magnetic particles disposed therein to accumulated plaque within a blood vessel; and
magnetically manipulating the ferrfluids to remove the accumulated plaque.

32. The method of claim 31, wherein the magnetic particles are dispersed in a carrier fluid within the ferrofluids.

33. The method of claim 32, wherein the carrier fluid is one of a water-based, a water-alcohol-based, or a hydrocarbon-based carrier fluid.

34. The method of claim 32, wherein the carrier fluid is hydrophilic.

35. The method of claim 32, wherein the carrier fluid is hydrophobic.

36. The method of claim 32, further comprising:
impregnating the ferrofluids with abrasive particles, the ferrofluids and abrasive particles combining to break-up and remove the accumulated plaque by the magnetic manipulation of the ferrofluids.

37. The method of claim 36, wherein the abrasive particles are displaced to a surface of the ferrofluids to help break-up and remove the accumulated plaque upon magnetic manipulation of the ferrofluids.

38. The method of claim 34, wherein the abrasive particles are hydrophobic and are displaced to a surface of the ferrofluids by the hydrophilic carrier fluid of the magnetic particles, the displaced abrasive particles and ferrofluids combining to help break-up and remove the accumulated plaque.

39. The method of claim 35, wherein the abrasive particles are hydrophilic and are displaced to a surface of the ferrofluids by the hydrophobic carrier fluid of the magnetic particles, the displaced abrasive particles and ferrofluids combining to help break-up and remove the accumulated plaque.

40. The method of claim 32, further comprising:
mixing an anti-plaque drug with the ferrofluids, the ferrofluids and anti-plaque drug combining to break-up and remove the accumulated plaque.

41. The method of claim 40, wherein the anti-plaque drug is bonded to the magnetic particles or the abrasive particles to mix with the ferrofluids.

42. The method of claim 40, wherein the anti-plaque drug is mixed with the carrier fluid of the magnetic particles or is added by carrier particles to mix with the ferrofluids.

43. The method of claim 32, further comprising:
impregnating the ferrofluids with a combination of the magnetic particles, the abrasive particles and an anti-plaque drug prior to delivering the ferrofluids to the accumulated plaque, the ferrofluids, abrasive particles and anti-plaque drug combining to break-up and remove the accumulated plaque.

44. The method of claim 31, further comprising:
creating a vortex, high velocity jets, or other motion with the ferrofluids by the magnetic manipulation of the ferrofluids, the vortex, high velocity jets or other motion aiding the removal of the accumulated plaque.

45. The method of claim 44, wherein the vortex, high velocity jets or other motion detaches the accumulated plaque from the blood vessel and the detached plaque is mechanically extracted therefrom the blood vessel.

46. The method of claim 44, wherein the vortex, high velocity jets or other motion breaks-up and removes the accumulated plaque from the blood vessel.

47. The method of claim 44, wherein the ferrofluids are delivered directly to the accumulated plaque using a catheter or a micro-catheter.

48. The method of claim 44, wherein the ferrofluids are delivered intravenously and then magnetically manipulated to concentrate at the accumulated plaque.

49. The method of claim 44, further comprising:
magnetically manipulating the ferrofluids using an external magnetic field generator into which at least a portion of a patient is placed, the magnetic field generator having a collar and at least one magnet that generates a magnetic field, the collar and the at least one magnet being positioned to externally encircle a body part whereat the accumulated plaque is located, whereby the collar and the at least one magnet creates the magnetic field that magnetically manipulates the ferrofluids to break-up and remove the accumulated plaque.

50. The method of claim 49, wherein the magnetic field manipulates the ferrofluids by at least one of rotating or moving the collar and the at least one magnet.

51. The method of claim 50, further comprising:
sensing the activity of the ferrofluids at the accumulated plaque using a magnetically tipped guide-wire having at least one sensor in the tip thereof.

52. The method of claim 50, determining the activity of the ferrofluids by sensing the amount of deflection of the magnetic guide-wire tip using the at least one sensor.

53. The method of claim 50, further comprising:
determining the activity of the ferrofluids at the accumulated plaque by sensing one or more of pressure, temperature, magnetic field strength, and magnetic field gradient using the at least one sensor.

54. The method of claim 50, wherein the magnetic field generator is a portable tubular member into which a body part having the accumulated plaque is placed to magnetically manipulate the ferrofluids.

55. The method of claim 44, further comprising:
magnetically concentrating the ferrofluids at the accumulated plaque using an external magnetic field generator into which at least a portion of a patient is placed, the magnetic field generator having a collar and at least one magnet that generates a magnetic field, the collar and the at least one magnet being positioned to externally encircle a body part whereat the accumulated plaque is located; and
magnetically manipulating the ferrofluids using the external magnetic field generator by rotating or moving the collar and the at least one magnet to create the magnetic field that manipulates the ferrofluids to break-up and remove the accumulated plaque.

56. The method of claim 49, further comprising sensing the activity of the ferrofluids at the accumulated plaque using at least one sensor at a tip of a catheter or a micro-catheter.

57. The method of claim 49, wherein the at least one magnet is a plurality of magnets independently operable to generate the magnetic field.

58. The method of claim 49, wherein the magnetic field generator or a position of the patient is altered to alter a gradient, geometry or strength of the magnetic field.
